(19) United States
(12) Patent Application Publication
(10) Pub. No.: US 2004/0255346 A1
(43) Pub. Date: Dec. 16, 2004

(54) Transgenic plants over-expressing a heat shock factor

(76) Inventors: Yee-Yung Charng, Taipei (TW); Ming-Tsair Chan, Taipei (TW); Hsiao-Yuan Li, Taipei (TW); Hui-Wen Tsai, Taipei (TW); Lin-Yun Kuang, Taipei (TW)

Correspondence Address: FISH & RICHARDSON PC, 225 FRANKLIN ST, BOSTON, MA, 02110, US

(21) Appl. No.: 10/461,609

(22) Filed: Jun. 13, 2003

(51) Int. Cl. C12N 15/82; C12N 15/87; A01H 5/00; C12N 5/10

(52) U.S. Cl. 800/278; 435/419; 435/468; 800/317.4

(57) ABSTRACT

A transformed plant cell or transgenic plant containing a recombinant nucleic acid that encodes a heterologous heat shock factor. Expression of the heat shock factor derepresses the heat shock response in the transformed plant cell or transgenic plant under non-heat shock conditions. Also disclosed are methods of producing such a transformed plant cell or transgenic plant.

### BACKGROUND

Heat treatment has been shown to protect against chilling injury in a number of plant species, including avocado (Woolf (1997) HortSci. 32:1247-1251), cucumber (Lafuente et al. (1991) Plant Physiol. 95:443-449 and McCollum et al. (1995) Postharvest Biol. Technol. 6:55-64), mango (McCollum et al. (1993) HortSci. 28:197-198), pepper (Mencarelli et al. (1993) Acta Hortic. 343:238-243), rice (Sato et al. (2001) J. Exp. Bot. 52:145-151), and tomato (Lurie and Klein (1991) J. Am. Soc. Hortic. Sci. 116:1007-1012 and Rab and Saltveit (1996) J. Am. Soc. Hort. Sci. 121:711-715). The heat-shock-induced chilling tolerance (HSICT) occurs in a wide range of plants. It has been shown that expression of small heat shock proteins (sHsps) is correlated to HSICT (Sabehat et al. (1998) Plant Physiol. 117:651-658). A heat-inducible ascorbate peroxidase gene APX, whose promoter contains a minimal heat-shock-element (HSE), has also been shown to be associated with HSICT in rice (Sato et al. (2001) J. Exp. Bot. 52:145-151). Hence, it is very likely that the expression of heat shock responsive genes is responsible for HSICT. As heat shock factors (Hsfs) play a major role in regulating the expression of the heat shock-responsive genes (Nover et al. (2001) Cell Stress Chaperones 6:177-189), over-expression of a transgenic Hsf may derepress the heat shock response and increase thermal- and chilling-tolerance in a transgenic plant.

### SUMMARY

This invention relates to expressing a heterologous heat shock factor in a plant cell, thereby derepressing the heat shock response in the cell under non-heat shock conditions.

In one aspect, the invention features a transformed plant cell containing a recombinant nucleic acid that encodes a heterologous heat shock factor. Expression of the heat shock factor derepresses the heat shock response in the transformed plant cell under non-heat shock conditions. The heat shock factors useful for this invention include, for example, Arabidopsis heat shock factor Alb. The plant cell can be a dicot plant cell, e.g., a tomato cell.

A transformed plant cell of this invention can be cultivated to generate a transgenic plant. Such a transgenic plant is within the scope of the invention. More specifically, the invention features a transgenic plant whose genome contains a recombinant nucleic acid encoding a heterologous heat shock factor. Expression of the heat shock factor derepresses the heat shock response in the plant under non-heat shock conditions.

A “recombinant nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein.

“Non-heat shock conditions,” as used herein, refers to temperatures at which the plant cell normally grows.

In another aspect, the invention features a method of producing a transformed plant cell. The method involves introducing into a plant cell a recombinant nucleic acid that encodes a heterologous heat shock factor and expressing the heat shock factor in the cell to derepress the heat shock response under non-heat shock conditions.

Also within the scope of this invention is a method of producing a transgenic plant. The method involves introducing into a plant cell a recombinant nucleic acid encoding a heterologous heat shock factor, expressing the heat shock factor in the cell, and cultivating the cell to generate a plant. Expression of the heat shock factor derepresses the heat shock response in the plant under non-heat shock conditions.

This invention provides a method of generating a transgenic plant with enhanced thermo- and chilling-tolerance. The details of some embodiments of the invention are set forth in the description below. Other features, objects, and advantages of the invention will be apparent from the description, and from the claims.

### DETAILED DESCRIPTION

Chilling injury is a physiological disorder of plant when exposed to nonfreezing temperatures below about 12° C. Many important crops indigenous to the tropics and subtropics, such as banana, mango, papaya, rice, and tomato, are susceptible to chilling injury. Chilling injury manifests a range of visible symptoms, which are often used as indicators of its severity, including reduced growth vigor, abnormal ripening, stimulated respiration and ethylene production, and increased cellular membrane leakage and disease susceptibility (Lyons (1973) Annu. Rev. Plant Physiol. 24: 445-466 and Saltveit and Morris (1990) Overview on chilling injury of horticultural crops. In CY Wang, ed., Chilling injury of horticultural crops. CRC Press, Boca Raton, Fla., pp 3-15). It has been shown that these symptoms can be reduced by heat shock treatment before exposure to chilling temperatures (Lafuente et al. (1991) Plant Physiol. 95:443-449, Lurie and Klein (1991) J. Am. Soc. Hortic. Sci. 116:1007-1012, Saltveit (1991) Physiol. Plant 82:529-536, McCollum et al. (1993) HortSci. 28:197-198, Collins et al. (1995) J. Exp. Bot. 46:795-802, Woolfet al. (1995) J. Am. Soc. Hortic. Sci. 120:1050-1056, Rab and Saltveit (1996) J. Am. Soc. Hort. Sci. 121:711-715, and Sato et al. (2001) J. Exp. Bot. 52:145-151), a phenomenon that has been termed heat-shock-induced chilling tolerance (HSICT). De novo protein synthesis seems to be necessary for HSICT in mung bean hypocotyls (Collins et al. (1995) J. Exp. Bot. 46:795-802). The exact mechanism of HSICT is not clear.

When organisms are exposed to increased high temperature, the induction of genes encoding heat shock proteins (HSPs) is one of the most prominent responses at the molecular level (Kimpel and Key (1985) Trends Biochem. Sci. 10:353-357, Lindquist (1986) Annu. Rev. Biochem. 55:1151-1191, Vierling (1991) Annu. Rev. Plant Physiol. Plant Mol. Biol. 42:579-620, and Waters et al. (1996) J. Exp. Bot. 47:325-338). In addition to HSPs, oxidative stress relieving enzymes, such as ascorbate peroxidase (APX) (Mittler and Zilinskas (1992) J. Biol. Chem. 267:21802-21807, Mittler and Zilinskas (1994) Plant J. 5:397-405, Storozhenko et al. (1998) Plant Physiol. 118:1005-1014, and Shi et al. (2001) Gene 273:23-27), was also found inducible by heat shock treatment.

In general, the regulation of expression of many HSP genes is mediated by the conserved heat shock factor (HSF). The latent HSF is activated upon heat treatment by induction of trimerization and high-affinity binding to the heat-shock-element (HSE), a conserved sequence present in the promoter regions of many HSP genes (Wu (1995) Annu. Rev. Cell Dev. Biol. 11:441-469). Over-expression of HSF has been shown to cause constitutive synthesis of HSPs and increased basal thermotolerance in transgenic Arabidopsis (Lee et al. (1995) Plant J. 8:603-612 and Prandl et al. (1998) Mol. Gen. Genet. 258:269-278). These results indicate that HSF plays a pivotal role in thermotolerance by regulating downstream HSP genes.

This invention is based on an unexpected discovery that expression of Arabidopsis thaliana HSF3 (AtHsfA1b) in tomato derepresses heat shock responses and increases thermal- and chilling-tolerance of the transgenic plant. This finding is useful for conferring chilling tolerance in post-harvest produce without the requirement of heat acclimation treatment and for improving traits against high temperature stress.

Accordingly, the invention features a transformed plant cell containing a recombinant nucleic acid that encodes a heterologous heat shock factor. Expression of the heat shock factor derepresses the heat shock response in the transformed plant cell under non-heat shock conditions. The heat shock factors useful for this invention include, for example, Arabidopsis heat shock factor A1a, A1b, A1d, A1e or tomato heat shock factor A1. The plant cell can be a dicot plant cell, e.g., a tomato cell, a mango cell, a cucumber cell or a pepper cell, or a monocot plant cell, e.g., a rice cell.

A transformed plant cell of the invention can be produced by introducing into a plant cell a recombinant nucleic acid that encodes a heterologous heat shock factor and expressing the heat shock factor in the cell to derepress the heat shock response under non-heat shock conditions.

Techniques for transforming a wide variety of plant cells are well known in the art and described in the technical and scientific literature. See, for example, Weising et al. (1988) Ann. Rev. Genet. 22:421-477. To express a heterologous heat shock factor gene in a plant cell, the gene can be combined with transcriptional and translational initiation regulatory sequences which will direct the transcription of the gene and translation of the encoded protein in the plant cell.

For example, for overexpression, a constitutive plant promoter may be employed. A “constitutive” promoter is active under most environmental conditions and states of cell differentiation. Examples of constitutive promoters include the cauliflower mosaic virus (CaMV) 35S transcription initiation region, the 1′- or 2′-promoter derived from T-DNA of Agrobacterium tumafaciens, the ACT11 and Cat3 promoters from Arabidopsis (Huang et al. (1996) Plant Mol. Biol. 33:125-139 and Zhong et al. (1996) Mol. Gen. Genet. 251:196-203), the stearoyl-acyl carrier protein desaturase gene promoter from Brassica napus (Solocombe et al. (1994) Plant Physiol. 104:1167-1176), the GPc1 and Gpc2 promoters from maize (Martinez et al. (1989) J. Mol. Biol. 208:551-565 and Manjunath et al. (1997) Plant Mol. Biol. 33:97-112).

Alternatively, a plant promoter may be employed to direct expression of the heat shock factor gene in a specific cell type (i.e., tissue-specific promoters) or under more precise environmental or developmental control (i.e., inducible promoters). Examples of environmental conditions that may affect transcription by inducible promoters include anaerobic conditions, elevated temperature, the presence of light, spray with chemicals or hormones, or infection of a pathogen. Examples of such promoters include the root-specific ANR1 promoter (Zhang and Forde (1998) Science 279:407) and the photosynthetic organ-specific RBCS promoter (Khoudi et al. (1997) Gene 197:343).

For proper polypeptide expression, a polyadenylation region at the 3′-end of the coding region should be included. The polyadenylation region can be derived from the natural gene, from a variety of other plant genes, or from T-DNA.

A marker gene can also be included to confer a selectable phenotype on plant cells. For example, the marker gene may encode a protein that confers biocide resistance, antibiotic resistance (e.g., resistance to kanamycin, G418, bleomycin, hygromycin), or herbicide resistance (e.g., resistance to chlorosulfuron or Basta).

A recombinant nucleic acid that encodes a heterologous heat shock factor may be introduced into the genome of a desired plant host cell by a variety of conventional techniques. For example, the recombinant nucleic acid may be introduced directly into the genomic DNA of a plant cell using techniques such as electroporation and microinjection of plant cell protoplasts, or the recombinant nucleic acid can be introduced directly to plant tissue using ballistic methods, such as DNA particle bombardment.

Microinjection techniques are known in the art and well described in the scientific and patent literature. The introduction of a recombinant nucleic acid using polyethylene glycol precipitation is described in Paszkowski et al. (1984) EMBO J. 3:2717-2722. Electroporation techniques are described in Fromm et al. (1985) Proc. Natl. Acad. Sci. USA 82:5824. Ballistic transformation techniques are described in Klein et al. (1987) Nature 327:70-73.

Alternatively, the recombinant nucleic acid may be combined with suitable T-DNA flanking regions and introduced into a conventional Agrobacterium tumefaciens host vector. The virulence functions of the Agrobacterium tumefaciens host will direct the insertion of the heat shock factor gene and adjacent marker into the plant cell DNA when the cell is infected by the bacteria. Agrobacterium tumefaciens-mediated transformation techniques, including disarming and use of binary vectors, are well described in the scientific literature. See, for example, Horsch et al. (1984) Science 233:496-498, Fraley et al. (1983) Proc. Natl. Acad. Sci. USA 80:4803, and Gene Transfer to Plants, Potrykus, ed., Springer-Verlag, Berlin, 1995.

The presence and copy number of the heterologous heat shock factor gene in a transgenic plant can be determined using methods well known in the art, e.g., Southern blotting analysis. Expression of the heterologous heat shock factor gene in a transgenic plant may be confirmed by detecting the heterologous heat shock factor mRNA or protein in the transgenic plant. Means for detecting and quantifying mRNA or proteins are well known in the art.

Transformed plant cells which are derived by any of the above transformation techniques can be cultured to regenerate a whole plant. Such regeneration techniques rely on manipulation of certain phytohormones in a tissue culture growth medium, typically relying on a biocide or herbicide marker that has been introduced together with the heat shock factor gene. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. (1987) Ann. Rev. Plant Phys. 38:467-486. Once the heterologous heat shock factor gene has been confirmed to be stably incorporated in the genome of a transgenic plant, it can be introduced into other plants by sexual crossing. Any of a number of standard breeding techniques can be used, depending upon the species to be crossed.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Materials and Methods

Expression Plasmid Constructions

The construct for expression of AtHsfA1b-GUS fusion protein was generated according to the method previously described by Prandl et al. ((1998) Mol. Gen. Genet. 258:269-278) with some modification. The AtHsfA1b (also named Hsf3) open reading frame (ORF) (GenBank accession number Y14068) amplified by reverse transcriptase polymerase chain reaction (RT-PCR) was fused to gusA in pBI221 instead of pBI121.1 (Prandl et al. (1998) Mol. Gen. Genet. 258:269-278) to yield pYC018. The ORF was sequenced to make sure no unwanted mutation had been generated by PCR. The HindIII-EcORI fragment from pYC018 containing the cauliflower mosaic virus 35S promoter, the AtHsfA1b-gusA fusion, and nos termination signal sequence was ligated into the corresponding sites of a binary vector, pCAMBIA2300 (Center for the Application of Molecular Biology of International Agriculture, Australia), and the resulting plasmid was designated as pYC019. Plasmid pCAMBIA2301 with a 35S-GUS-Nos construct was employed to transform tomato as a control. The plasmids were introduced into Agrobacterium tumefaciens strain LBA4404 by the freeze-thaw method (Holsters et al. (1978) Mol. Gen. Genet. 163:181-187).

Generation and Growth of Transgenic Tomato Plants

A local tomato inbred line, L4783, provided by the Asian Vegetable Research and Development Center, Tainan, Taiwan, was employed for genetic transformation. Tomato transformation was performed according to the method of Fillatti et al. ((1987) Plant Biol. 4:199-210) and the medium was prepared according to Hamza and Chupeau ((1993) J. Exp. Bot. 44:1837-1845) with slight modification. Cotyledons from 7- to 8-day-old seedlings were cut into two pieces, placed upside down on the pre-culture medium (MS salts, Gamburg's B5 vitamins, 2 mg/L BA, 0.25 mg/L IAA, and 0.3% Phytagel), and then incubated in dark for 24 h before inoculation with A. tumefaciens cells harboring either pYCO 9 or pCAMBIA230 1. The explants were submerged in Agrobacterium inoculum for 30 min, blotted dry, transferred to the co-cultivation medium (the pre-culture medium supplemented with 200 μM acetosyringone) and incubated in dark for 2 days. Following co-cultivation, the explants were transferred to the AZ medium (Hamza and Chupeau (1993) J. Exp. Bot. 44:1837-1845) supplemented with 100 mg/L kanamycin and 200 mg/L Timentin (Duchefa Biochemie BV, the Netherlands) for regeneration and selection. Three to four weeks later, explants with developing shoots were transferred to a shoot elongation medium which was the same as the AZ medium except that zeatin and IAA were excluded. When the shoots grew to 2-3 cm tall, they were transferred to the MM medium (Hamza and Chupeau (1993) J. Exp. Bot. 44:1837-1845) with 50 mg/L kanamycin for rooting before they were transferred to potting media. Transgenic and wild-type tomatoes were grown in a green house facility during the October to May period when average temperature was maintained at less than 25° C. during the day and less than 20° C. during the night.

Analysis of Transgenic Tomato Plants

For comparative analysis of transgenic and wild-type plants in each experiment, the samples were harvested from the same batch of plants grown at the same time. For Southern blot analysis of putative transgenic plants, about 10 μg of purified genomic DNA was digested with restriction enzymes and resolved on a 0.6% agarose gel in 0.5×TBE buffer (Sambrook et al. ((1989) Molecular Cloning: A Laboratory Manual , Ed. 2. Cold Spring Harbor Laboratory Press, Cold Spring Harbor, N.Y.). Capillary blotting of the DNA from the gel to a positively charged nylon membrane (Magnacharge, MSI) was performed according to Sambrook et al. ((1989) Molecular Cloning: A Laboratory Manual , Ed. 2. Cold Spring Harbor Laboratory Press, Cold Spring Harbor, N.Y.). To generate a non-radioactive hybridization probe for detection of the transgene, a 535 bp DNA fragment corresponding to nucleotides 860-1394 of the AtHsfA1b ORF (Prandl et al. (1998) Mol. Gen. Genet. 258:269-278) was produced by PCR and labeled with a PCR DIG-labeling kit (Roche). Hybridization and washing were performed following the manufacturer's protocol. For Northern blot analysis, total RNA was isolated from plant tissues using a commercial reagent according to the manufacturer's instructions (TRIZOL, Gibco BRL). Separation of RNA on a formaldehyde-1% agarose gel (20 μg of RNA per lane) and transfer of RNA to a positively charged nylon membrane (Magnacharge, MSI) by capillary blotting were performed according to Sambrook et al. ((1989) Molecular Cloning: A Laboratory Manual, Ed. 2. Cold Spring Harbor Laboratory Press, Cold Spring Harbor, N.Y.). A 535 bp DNA fragment corresponding to nucleotides 860-1394 of the AtHsfA1b ORF, cDNA corresponding to tomato full-length ORF of Hsp17.8-CI (GenBank accession number X56138), and cDNA corresponding to tomato full-length ORF of Hsp26.1-P (GenBank accession number U59917) were amplified by RT-PCR with gene-specific primers and individually cloned into pGEM T-easy (Promega) vector for producing DIG-labeled anti-sense RNA probes. The probe for tomato Hsp70 was derived from a tomato EST clone (TIGR Tomato Gene Index AW223426) that shares 92% identity with the tobacco Hsp70 cDNA (GenBank accession number X63106). DIG-labeled anti-sense RNA probes were produced by in vitro transcription using SP6 or T7 RNA polymerase (Promega) and DIG-RNA probe mix (Roche) according to the manufacturer's instructions. Pre-hybridization (4 h) and hybridization (16 h) were performed at 65° C. in a solution containing 50% formamide and 50 ng/mL of a DIG-labelled probe. Membranes were washed twice in 2×SSC (Sambrook et al. (1989) Molecular Cloning: A Laboratory Manual, Ed. 2. Cold Spring Harbor Laboratory Press, Cold Spring Harbor, N.Y.) and 0.1% SDS at room temperature and twice in 0.1×SSC and 0.1% SDS at 65° C. for 15 min each. The amount of hybridized DIG-labelled probe on the membrane was determined according to the manufacturer's protocol.

Enzyme Activity Assays

Histochemical staining and fluorometric assay of GUS activity were performed according to the method of Jefferson ((1987) Plant Mol. Biol. Rep. 5:387-405). For APX and catalase assays, the six-day-old etiolated seedlings were immediately frozen in liquid nitrogen after treatment and ground into fine powder with mortar and pestle and homogenized in a cold sodium phosphate buffer (50 mM, pH 7.0) with a homogenizer. The crude extract was centrifuged at 4° C. and 12,000×g for 20 min. The supernatant was used immediately for enzyme assays. Activities of soluble isoforms of APX were determined using a spectrophotometry method as previously described (Nakano and Asada (1981) Plant Cell Physiol. 22:867-880). Catalase activity was analyzed according to the method of Kato and Shimizu ((1987) Can. J Bot. 65:729-735). Superoxide dismutase (SOD) isozyme activities were determined by an in gel assay method according to Chen and Pan ((1996) Acad. Sin. 37:107-111). The amount of protein was measured according to the dye-binding method (Bradford (1976) Anal. Biochem. 72:248-254) using bovine serum albumin as the standard.

Immunoblotting Analysis

SDS-PAGE was performed on 4-12% Bis-Tris precast gels (Invitrogen). After electrophoresis, proteins were electrophoretically transferred to a nitrocellulose membrane. The membrane was blocked in PBS buffer (potassium phosphate 25 mM, NaCl 150 mM, pH 7.2) with 5% skim milk and 0.2% Tween-20, and probed with 10,000-fold diluted polyclonal antibody raised against rice class I sHsp (Jinn et al. (1993) Plant Cell Physiol. 34:1055-1062), which was kindly provided by Prof. Chu-Yung Lin of National Taiwan University. Following secondary antibody reaction employing goat anti-rabbit IgG conjugated to horseradish peroxidase, the blots were visualized using the Super Signal West Dura Extended Duration Substrate system (Pierce).

Thermotolerance Test

The thermotolerance of tomato seedlings was measured by adopting the method of Burke ((1994) In J H Cherry, ed., Biochemical and Cellular Mechanisms of Stress Tolerance in Plants. Springer-Verlag, Berlin, pp 191-200). Tomato seeds were surface-sterilized and germinated in dark at 25° C. on the GM medium (MS salt, 2% sucrose, and 0.3% phytagel) in a glass jar (400 mL) capped with a glass petri dish cover. In each jar, 10 seeds were included. Six-day-old etiolated seedlings were then subjected to heat treatment at a lethal temperature, 50° C. for 2 h, either with or without prior heat acclimation treatment. For heat acclimation, the samples were placed at 40° C. for 2 h and recovered at 25° C. for 1 h before further treatment. All the above treatments were performed by placing the jars in a controlled temperature oven for an indicated length of time, and light was avoided. The air temperature inside the jar reached desired level within 15 min as monitored by a thermometer. Following heat treatment, seedlings were exposed to white light (150 μmol m⁻² s⁻¹) under light/dark cycle (16 h/8 h) at 25° C. for two days to allow chlorophyll synthesis. After photographing the seedlings, the cotyledons were harvested and the chlorophyll content was measured according to the method described previously (Moran and Porath (1980) Plant Physiol. 65:478-479).

Chilling-Tolerance Test

The chilling tolerance of tomato seedlings was tested by measuring radicle length or survival rate following chilling treatment. The former study was performed basically according to the method of Rab and Sailveit ((1996) J. Am. Soc. Hort. Sci. 121:711-715) with some modification. T₃ seeds of homozygous transgenic lines and wild-type tomato were soaked in distilled water overnight with gentle shaking at 25° C., and transferred to 3 layers of wet paper towel between two 19×19.5 cm glass plates for germination. The germinated seeds with about 5 mm radicle lengths were picked out and incubated at 25° C. or 38° C. for 15 min in a water bath. The seedlings were then positioned back into the paper towel and glass plate sandwich for chilling treatment, which was set at 2.5° C. for 5 days in dark, and transferred to 25° C. for 3 days also in dark for continued growth of seedlings. Subsequent radical elongation was measured and recorded at the end of the 3-day regrowth period. To measure the survival rate, 3-day-old seedlings were subjected to storage in dark at 2.5° C. for 7-18 days on moisten 3MM filter paper. For heat shock acclimation, the seedlings were first incubated at 38° C. for 15 min before directly subjected to chilling treatment. Following low temperature treatment, the seedlings were transferred to a 25° C. incubator for 6 days under light. Seedlings shown green cotyledons and adventitious roots were counted as survivors. Survival rate was calculated according to the following equation: (number of survivors/number of tested seedlings)×100.

Results

Identification of Transgenic Tomato Plants Harboring AtHsfA1b-gusA

To evaluate the effect of expression of Hsf on chilling tolerance in tomato, the fused AtHsfA1b-gusA transgene was introduced into tomato under the control of cauliflower mosaic virus 35S promoter using Agrobacterium-mediated transformation. The GUS fusion protein was introduced as a reporter for easy and sensitive identification of the transgene. The same AtHsfA1b-gusA chimeric gene has previously been shown to encode an AtHsfA1b-GUS fusion protein with full Hsf activity (Prandl et al. (1998) Mol. Gen. Genet. 258:269-278). Transgenic tomato plants containing only the 35S::gusA transgene were also generated using the same method and employed as a control.

Thirty-four independent Ro transgenic AtHsfA1b-gusA plants were generated and analyzed by histochemical staining of GUS activity and Southern blot analyses to determine the presence, genomic integration, and insertion copy number of AtHsfA1b-gusA. Gene silencing was observed for some of the To transgenic plants with multiple insertions of the transgene. Three independent transgenic plants, TT1-15, TT1-20 and TT1-22, each with a single insertion and different expression level of AtHsfA1b-gusA were selected for further studies. These transgenic plants were allowed to self-pollinate to generate plants homozygous for AtHsfA1b-gusA. All of the T₁ transgenic plants showed an approximate 3:1 segregation ratio based on the GUS activity assay. This observation agreed well with the result of Southern blot analysis showing that the foreign gene was integrated into a single genetic locus.

According to GUS histochemical staining of the test transgenic plants, the transgene was expressed in cotyledon, root, young leaf, stem, and fruit tissues. Histochemical staining for GUS activity showed intensified GUS staining at the site of nuclei in AtHsfA1b-gusA transgenic lines, which was not observed for gusA transgenic plants. This staining pattern indicates that AtHsfA1b-gusA product was localized within the nuclei in the absence of heat shock treatment.

Over-Expression of AtHsfA1b Derepressed Heat Shock Response in Transgenic Tomato Plants

Over-expression of AtHsfA1b-gusA in Arabidopsis derepresses heat shock response (Prandl et al. (1998) Mol. Gen. Genet. 258:269-278). Transcript levels of three Hsp genes, Hsp17.8-CI, Hsp26.1-P, and Hsp70 were measured in immature green tomato fruits (about 30 days after anthesis) to determine whether expression of AtHsfA1b-gusA in transgenic tomato plants also conferred similar effects. Heat treatment up-regulates Hsp17.8-CI (also named tom66) and Hsp26.1-P (also named tom111), and class I cytosolic and chloroplast sHsp in mature green tomato fruit (Sabehat et al. (1998) Plant Physiol. 117:651-658). Atomato EST (Expressed Sequence Tags) clone (AW223426) that shares 92% identity with the tobacco Hsp70 cDNA (GenBank accession number X63106), designated as Hsp70, was also induced in heat-treated fruits.

AtHsfA1b-gusA transcripts were readily detected in transgenic plants using Northern blot analysis, the level being higher in TT1-15 and -20 than in TT1-22. Under non-heat shock conditions, the transcripts of Hsp26.1-P and Hsp70 accumulated in fruits of transgenic lines TT1-15 and 20 at a higher level than in wild-type plants or plants transformed with the gusA gene only. In contrast, TT1-22 showed little difference. However, the transcript levels of Hsp17.8-CI, a class I sHsp, in TT1-20 and 22 were about the same or even lower than in the wild-type plant. There are at least four homologous genes that share 90-99% identity with Hsp17.8-CI in the coding region according to the data in TIGR Tomato Gene Index database. It is likely that the probe derived from HSP17.8-C1 cross-hybridizes to the transcripts of one or more of these homologous genes.

The amount of class I sHsp protein in the immature green fruits was determined by Western blot analysis using polyclonal antibodies raised against rice class I sHsp (Jinn et al. (1993) Plant Cell Physiol. 34:1055-1062). The antibodies specifically recognized a heat-inducible protein of 20 kDa as determined from its mobility on SDS-PAGE, which was close to the calculated size of Hsp17.8-C1. This suggests that the protein recognized by the antibodies was the product encoded by Hsp17.8-CI, or at least by its homolog. The class I sHsp could be detected in all the transgenic lines, even without heat treatment, but they could not be detected in wild-type plants or plants transformed with gusA gene expression only. Although the transcript levels of Hsp17.8-CI in TT1-20 and TT1-22 were not higher than in the wild-type plants, the class I sHsp accumulated to a level significantly higher than in the wild-type plants. Taken together, these results indicate that high-level expression of AtHsfA1b-gusA led to the expression of Hsps in transgenic tomato plants, even in the absence of heat shock treatment.

The transcript levels of Hsps, however, did not increase in the cotyledons of the AtHsfA1b-gusA transgenic seedlings as they did in immature green fruits under non-stressed conditions. Although the transcript levels of AtHsfA1b in the cotyledons of 6-day-old etiolated seedlings were between 1.5- to 2-fold higher than in the fruit tissue, the transcript levels of Hsp17.8-CI, Hsp26.1-P and Hsp 70 were almost undetectable in the cotyledon of the transgenic plants under non-heat shock conditions. Although the transcript of Hsp17.8-CI was not detectable in the cotyledons of the transgenic AtHsfA1b-gusA plants, the class I sHsp significantly accumulated to about the same level as in the fruit tissue. Such discrepancy between sHsp transcripts and protein levels has been previously reported between heat-shocked callus and somatic embryo cells of carrot (Zimmerman et al. (1989) Plant Cell 1:1137-1146). These results suggest that effective expression of AtHsfA1b-gusA derepressed heat shock response in different parts of the transgenic plants, i.e., in fruit and in seedlings. Therefore, the subsequent tests were limited to seedlings in the examination of the effect of over-expression of AtHsfA1b-gusA on heat and chilling tolerance.

Despite of the constitutive heat shock response, the transgenic AtHsfA1 b-gusA plants did not show significant alteration in growth or morphology and produced normal fruits and viable seeds under non-stressed growth conditions.

Higher Ascorbate Peroxidase Activity in Transgenic Tomato Plants

A heat-inducible cytosolic APX is thought to be involved in HSICT in rice seedling (Sato et al. (2001) J. Exp. Bot. 52:145-151). The rice APX gene promoter contains a minimal heat shock factor-binding motif, 5′-nGAAnnTTCn-3′, the so-called heat shock element (HSE) (Sato et al. (2001) J. Exp. Bot. 52:145-151). InArabidopsis, an HSE found in the APX1 promoter was shown to be recognized by the tomato Hsf in vitro and responsible for the in vivo heat-shock induction of the gene (Storozhenko et al. (1998) Plant Physiol. 118:1005-1014). Although it is not known whether tomato APX genes contain HSE in their promoters, whether APX activity was affected in the transgenic tomato plants was investigated. APX activity was found to increase by about 1.4- to 1.8-fold in the gusA transgenic and wild-type tomato etiolated seedlings, respectively, after heat shock treatment. This is consistent with the observation in rice that APX activity is up-regulated by heat stress (Sato et al. (2001) J. Exp. Bot. 52:145-151). Under non-heat shock conditions, the AtHsfA1b-gusA transgenic plants exhibited up to 2-fold higher APX activity than the wild-type or gusA transgenic plants. Heat shock treatment further increased APX activities in the AtHsfA1b-gusA transgenic plants. Since superoxide dismutase (SOD) and catalase are enzymes also involved in scavenging active oxygen species like APX, the activity levels of these two enzymes were also examined. There was no significant difference in either SOD or catalase activities between the transgenic and wild-type seedlings under non-heat shock or heat shock conditions, suggesting that these enzyme activities were not affected by constitutively expressed Hsf.

Constitutive Expression of AtHsfA1b Increased Basal Thermotolerance in Transgenic Tomato Seedlings

The thermotolerance of the transgenic tomatoes was evaluated to determine whether AtHsfA1b could function normally in a heterogenous host. To do so, a sensitive bioassay developed by Burke ((1994) In J H Cherry, ed., Biochemical and Cellular Mechanisms of Stress Tolerance in Plants. Springer-Verlag, Berlin, pp 191-200) was adopted. This bioassay method is based on the level of inhibition of chlorophyll accumulation in etiolated seedlings following challenges at lethally high temperatures. After exposure to 50° C. for 2 h and then recovery at 25° C. for 2 d, the cotyledons of the test etiolated seedlings of the wild-type plants or plants transformed with only a gusA transgene were not able to expand or turn green, and more than 50% of the tested seedlings failed to grow. However, if the seedlings were first exposed to a mild heat stress condition at 40° C. for 2 h before subject to the lethal temperature treatment, their cotyledons obviously became greenish, and all of the seedlings were able to continue to grow, a phenomenon that has been defined as heat acclimation or acquired thermotolerance. It was found that, without heat acclimation, all of the T₃ seedlings of the AtHsfA1b-gusA transgenic lines exhibited a better thermotolerance than the non-acclimated wild-type or gusA transgenic plants as evidenced by expanded and greenish cotyledons and continuing growth after treatment at 50° C. for 2 h.

Measurement of chlorophyll content of test seedlings allowed quantitative comparison of the effect. The data agreed well with the observation that the AtHsfA1b-gusA transgenic lines in general accumulated a significantly higher level of chlorophyll after heat treatment without acclimation, whereas heat acclimation further increased the chlorophyll accumulation rate. When tested at higher temperatures, the non-acclimated AtHsfA1b-gusA transgenic lines survived heat treatment up to 52° C. but all died at temperature above 54° C. Heat acclimation at 40° C. for 2 h further enhanced thermotolerance of the transgenic and the wild-type plants up to 54° C.

When the test was performed earlier in the T₁ generation, some of the T₁ seedlings of the AtHsfA1b-gusA transgenic lines showed no detectable GUS activity or enhanced thermotolerance due to segregation of the introduced transgenes in the T₁ generation, indicating that the AtHsfA1b-gusA transgene was responsible for the thermotolerance in transgenic tomato. The increased basal thermotolerance of the AtHsfA1b-gusA transgenic plants, in addition to the constitutive heat shock response, suggest that the heterologous gene functioned normally in transgenic tomato.

Constitutive Expression of AtHsfA1b Improved Chilling Tolerance in Transgenic Tomato Seedlings

Class A Hsf is the major transcription regulator of heat shock response known to date. The effect of over-expressed AtHsfA1b-gusA on chilling tolerance in transgenic plants was evaluated. Inhibition of tomato seedling radicle growth by chilling treatment was measured according to the method of Rab and Saltveit ((1996) J. Am. Soc. Hort. Sci. 121:711-715). After recovery from storage at 2.5° C. for 5 days, the radicle growth of the wild-type and transgenic gusA plants were significantly reduced as compared to that without chilling treatment. Heat shock at 38° C. for 15 min prior to the chilling treatment was able to ameliorate the growth inhibition resulted from testing chilling stress. In the absence of heat shock pre-treatment, the AtHsfA1b-gusA plants exhibited a significantly better growth rate than the non-acclimated wild-type or gusA transgenic plants. Following chilling treatment, the levels of class I cytosolic sHsp, Hsp17.8-CI and APX activities remained high in the AtHsfA1b-gusA transgenic plants. Chilling treatment seemed to moderately induce synthesis of Hsp17.8-CI in the wild-type and gusA transgenic plants.

The survival rate of seedlings was then examined following a longer term of chilling treatment of 3-day-old seedlings, from 7 to 18 days. After recovery at 25° C. for 6 days of culture, the survivors were manifested by having expanding greenish cotyledons and adventitious roots. The wild-type plants generally did not endure the prolonged low temperature storage, and almost all seedlings died after 15 days of treatment. However, mild heat shock treatment applied immediately before the chilling treatment improved the survival rate by up to 30%. The AtHsfA1b-gusA transgenic plants were generally more tolerant to the chilling treatment than the wild-type plants without prior heat acclimation. The transgenic plants maintained 30-60% survival rate while the wild-type plants were all dead after 18 days of chilling treatment. Taken together, it was concluded that over-expression of the AtHsfA1b gene enhanced chilling tolerance of the transgenic plants due to derepression of heat shock responses.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A transformed plant cell comprising a recombinant nucleic acid that encodes a heterologous heat shock factor, wherein expression of the heat shock factor derepresses a heat shock response in the transformed plant cell under non-heat shock conditions.

2. The transformed plant cell of claim 1, wherein the heat shock factor is an Arabidopsis heat shock factor.

3. The transformed plant cell of claim 2, wherein the heat shock factor is a class A Arabidopsis heat shock factor.

4. The transformed plant cell of claim 3, wherein the heat shock factor is Arabidopsis heat shock factor A1b.

5. The transformed plant cell of claim 1, wherein the transformed plant cell is a dicot plant cell.

6. The transformed plant cell of claim 5, wherein the heat shock factor is an Arabidopsis heat shock factor.

7. The transformed plant cell of claim 6, wherein the heat shock factor is a class A Arabidopsis heat shock factor.

8. The transformed plant cell of claim 7, wherein the heat shock factor is Arabidopsis heat shock factor A1b.

9. The transformed plant cell of claim 5, wherein the transformed plant cell is a tomato cell.

10. The transformed plant cell of claim 9, wherein the heat shock factor is an Arabidopsis heat shock factor.

11. The transformed plant cell of claim 10, wherein the heat shock factor is a class A Arabidopsis heat shock factor.

12. The transformed plant cell of claim 11, wherein the heat shock factor is Arabidopsis heat shock factor Alb.

13. A transgenic plant whose genome comprises a recombinant nucleic acid encoding a heterologous heat shock factor, wherein expression of the heat shock factor derepresses a heat shock response in the transgenic plant under non-heat shock conditions.

14. The transgenic plant of claim 13, wherein the heat shock factor is an Arabidopsis heat shock factor.

15. The transgenic plant of claim 14, wherein the heat shock factor is a class A Arabidopsis heat shock factor.

16. The transgenic plant of claim 15, wherein the heat shock factor is Arabidopsis heat shock factor Alb.

17. The transgenic plant of claim 13, wherein the transgenic plant is a dicot plant.

18. The transgenic plant of claim 17, wherein the heat shock factor is an Arabidopsis heat shock factor.

19. The transgenic plant of claim 18, wherein the heat shock factor is a class A Arabidopsis heat shock factor.

20. The transgenic plant of claim 19, wherein the heat shock factor is Arabidopsis heat shock factor A1b.

21. The transgenic plant of claim 17, wherein the transgenic plant is tomato.

22. The transgenic plant of claim 21, wherein the heat shock factor is an Arabidopsis heat shock factor.

23. The transgenic plant of claim 22, wherein the heat shock factor is a class A Arabidopsis heat shock factor.

24. The transgenic plant of claim 23, wherein the heat shock factor is Arabidopsis heat shock factor A1b.

25. A method of producing a transformed plant cell, the method comprising:
introducing into a plant cell a recombinant nucleic acid that encodes a heterologous heat shock factor, and
expressing the heat shock factor in the cell,
wherein expression of the heat shock factor derepresses a heat shock response in the cell under non-heat shock conditions.

26. The method of claim 25, wherein the heat shock factor is an Arabidopsis heat shock factor.

27. The method of claim 25, wherein the plant cell is a dicot plant cell.

28. The method of claim 27, wherein the heat shock factor is an Arabidopsis heat shock factor.

29. The method of claim 27, wherein the plant cell is a tomato cell.

30. The method of claim 29, wherein the heat shock factor is an Arabidopsis heat shock factor.

31. A method of producing a transgenic plant, the method comprising:
introducing into a plant cell a recombinant nucleic acid encoding a heterologous heat shock factor,
expressing the heat shock factor in the cell, and
cultivating the cell to generate a plant,
wherein expression of the heat shock factor derepresses a heat shock response in the transgenic plant under non-heat shock conditions.

32. The method of claim 31, wherein the heat shock factor is an Arabidopsis heat shock factor.

33. The method of claim 31, wherein the transgenic plant is a dicot plant.

34. The method of claim 33, wherein the heat shock factor is an Arabidopsis heat shock factor.

35. The method of claim 33, wherein the transgenic plant is tomato.

36. The method of claim 35, wherein the heat shock factor is an Arabidopsis heat shock factor.
